(19) United States
(12) Patent Application Publication
(10) Pub. No.: US 2013/0116579 A1
(43) Pub. Date: May 9, 2013

(54) MEDICAL SYSTEM, AND A METHOD IN RELATION TO THE MEDICAL SYSTEM

(71) Applicant: ST. Jude Medical Systems AB, Uppsala (SE)

(72) Inventor: Johan SVANERUDH, Uppsala (SE)

(73) Assignee: ST. JUDE MEDICAL SYSTEMS AB, Uppsala (SE)

(21) Appl. No.: 13/661,260

(22) Filed: Oct. 26, 2012

Related U.S. Application Data
(60) Provisional application No. 61/552,563, filed on Oct. 28, 2011.

(51) Int. Cl. A61B 5/0215

(52) U.S. Cl. CPC A61B 5/0215
USPC 600/486

(57) ABSTRACT

A medical system for determining the individual Fractional Flow Reserve (FFR) value for one or many lesions of interest of a blood vessel, the system comprising an intravascular pressure measurement device for acquiring pressure measurements in the blood vessel during continuous blood flow there through. The pressure measurement device comprises a pressure sensor at its distal portion. The system further comprises an FFR processor adapted to determine the FFR value related to said lesion solely/only based upon the pressure measurements performed by said pressure sensor.

### FIELD OF THE INVENTION

The present invention relates to a medical system, and to a method in relation to the medical system, according to the preambles of the independent claims. In particular the invention relates to a medical system including a pressure measurement system adapted to determine the Fractional Flow Reserve value.

### BACKGROUND OF THE INVENTION

In many medical procedures, various physiological conditions present within a body cavity need to be monitored. These physiological conditions are typically physical in nature—such as pressure, temperature, rate-of-fluid flow, and provide the physician or medical technician with critical information as to the status of a patient's condition.

One device that is widely used to monitor conditions is the blood pressure sensor. A blood pressure sensor senses the magnitude of a patient's blood pressure, and converts it into a representative signal that is transmitted to the exterior of the patient.

In the prior art, it is known to mount a sensor at a distal portion of a so-called sensor wire and to position the sensor by using the sensor wire in a blood vessel in a living body to detect a physical parameter, such as pressure or temperature. The sensor includes elements that are directly or indirectly sensitive to the parameter.

One known sensor wire has a typical length of 1.5-2 meter, and comprises a hollow tubing running along a major part of the wire and having an outer diameter in the range of 0.25-0.5 mm, typically approximately 0.35 mm. A core wire is arranged within the tubing and extends along the tubing and often extends out from a distal opening of the tubing. The sensor or sensors is/are preferably arranged in connection with the distal portion of the core wire, e.g. at the distal end of the sensor wire.

The present invention is applicable e.g. in relation with a sensor wire of the type described above, but can also be applied to other types of sensor assemblies, e.g. where the sensor is arranged at the distal end of a catheter.

In one application the sensor wire of the type described above is used to measure pressure in blood vessels, and in particular in the coronary vessels of the heart, e.g. to identify constrictions in the coronary vessels. This may be performed by determining the so-called Fractional Flow Reserve (FFR) value related to the vessel. The sensor wire is typically inserted by use of an insertion catheter, which in turn is inserted via the femoral vein or the radial artery, and guided by the inserted catheter to the measurement site.

In order to power the sensor and to communicate signals representing the measured physiological variable to an external physiology monitor, one or more cables or leads, often denoted microcables, or optical cables, for transmitting the signals are connected to the sensor, and are routed along the sensor wire to be passed out from the vessel to the external physiology monitor, via physical cables or wirelessly.

The sensor element further comprises an electrical circuitry, which generally is connected in a Wheatstone bridge-type of arrangement to one or several piezoresistive elements provided on a membrane. As is well known in the art, a certain pressure exerted on the membrane from the surrounding medium will thereby correspond to a certain stretching or deflection of the membrane and thereby to a certain resistance of the piezoresistive elements mounted thereon and, in turn, to a certain output from the sensor element.

In US-2006/0009817, which is incorporated herein in its entirety, and which is assigned to the present assignee, an example of such a sensor and guide wire assembly is disclosed. The system comprises a sensor arranged to be disposed in the body, a control unit arranged to be disposed outside the body and a wired connection between the sensor and the control unit, to provide a supply voltage from the control unit to the sensor and to communicate a signal there between. The control unit further has a modulator, for modulating the received sensor signal and a communication interface for wireless communication of the modulated signal.

In U.S. Pat. No. 7,724,148, which is incorporated herein in its entirety, and which also is assigned to the present assignee, another example of such a pressure measurement system is disclosed. The pressure sensor wire is adapted to be connected, at its proximal end, to a transceiver unit that is adapted to wirelessly communicate via a communication signal with a communication unit arranged in connection with an external device.

In U.S. Pat. No. 6,112,598, which is incorporated herein in its entirety, and assigned to the present assignee, and also in U.S. Pat. No. 7,207,227, further examples of such pressure sensors and guide wire assemblies are disclosed.

As briefly discussed above, the human vascular system may suffer from a number of problems. These may broadly be characterised as cardiovascular and peripheral vascular disease. Among the types of disease, atherosclerosis is a particular problem. Atherosclerotic plaque can develop in a patient's cardiovascular system. The plaque can be quite extensive and occlude a substantial length of the vessel.

A technique used to identify and measure the extent of a stricture, also denoted lesion, caused by plaque is to measure the pressure inside the vessel in the part of the vessel where the stricture is located. In the prior art there are numerous examples of catheters suitable to perform pressure measurements. Among those may be mentioned U.S. Pat. No. 6,615,667 related to a guidewire provided with a combined flow, pressure and temperature sensor.

U.S. Pat. No. 6,565,514 relates to an exemplary measurement system adapted to measure, calculate and display physiological variables related to blood pressure and in particular for calculating the myocardial fractional flow reserve (FFR) being the ratio between the arterial pressure (Pa) and the distal coronary pressure (Pd).

US-2006/0052700 relates to a pressure measurement system comprising a pressure sensor guidewire provided with a pressure sensor at its distal end. The guidewire is adapted to be inserted into a vessel. The sensor is adapted to be drawn continuously along a section of the vessel under examination, e.g. by a pull-back device, and the recorded pressure data is mapped on a displayed image of the vessel.

Thus, FFR is a measure of coronary lesion severity and is defined as the ratio between distal and aortic blood pressure during maximum hyperemia. In all known systems on the market FFR is calculated using simultaneous pressure readings from two transducers. A pressure guide wire provided with a distal pressure sensor at its distal end which is arranged to measure the pressure at a position distal to the lesion, and a fluid filled pressure catheter connected to an external pressure transducer.

Any FFR measurement system using this setup must allow for connection of both pressure transducers which makes the system design and setup complicated since the external pressure transducer must also be connected to the cathlab's own recording system. Any pressure difference between the two transducers must also be removed at the start of the procedure through an equalization procedure.

In view of the above reasoning, the inventor has identified a need for a less complicated but still a reliable system and method for determining FFR.

### SUMMARY OF THE INVENTION

The above-mentioned object is achieved by the present invention according to the independent claims.

Preferred embodiments are set forth in the dependent claims.

The present invention relates to a system which only uses the pressure measurements from a single pressure transducer on a pressure guidewire or catheter as source for both the distal and the aortic pressures, enabling FFR calculation using only one transducer.

FFR is measured during maximum hyperemia.

If hyperemia is induced using continuous intravenous adenosine infusion the ratio between distal and aortic pressure is stable as long as the infusion is running. Utilizing this steady state, FFR can be assessed by the system by first recording a pressure from a position distally to the lesion and to the aortic root, pulling back the transducer and then using the pressure recorded by the same transducer now located in the aorta at the end of the pullback as reference pressure for the whole pullback. In this way FFR can be calculated by the system at the end of the pullback, assuming that the aortic pressure at the end of the pullback is representative for the whole pullback. In other words, a presumption is that the two measured pressure values are obtained essentially during similar states, i.e. the time interval between the points of time when the measurements are performed must not be too long, such that the aortic pressure is relatively stable during the pullback procedure.

Thus, the present invention relates to a medical system and a method for FFR measurement using only one pressure transducer.

The system and method offer a number of advantages.

Among those may be mentioned that there is no need for connection to an aortic pressure transducer in order to perform the FFR measurements, and therefore there is no need for any distal/proximal pressure equalization.

It is a single transducer system, preferably without connection to any catheter-lab environment.

Thus, the present invention simplifies the procedure of determining FFR.

In one advantageous setup the medical system and method according to the present invention may be used in combination with the wireless PressureWire™ Aeris™ (trademark owned by the present assignee) system where the proximal end of the pressure wire is connected to a transceiver unit that wirelessly communicates to a remote physiology monitor or a standard PC. Then a low cost, off the shelf, FFR measurement system is achieved. In such a set-up it would also be possible to present the FFR-value at a display provided at the transceiver unit.

The present invention is generally applicable for any type of pressure measurement device that comprises a pressure sensor at its distal portion. The pressure measurement device may e.g. be a pressure wire of the type referred to above, or a thin catheter provided with a pressure sensor at its distal portion, or a rapid-exchange catheter with a pressure sensor at its distal end, to mention some examples.

In one embodiment the pressure sensor comprises a piezoresistive element provided on a membrane on a chip arranged at the distal portion of the pressure measurement device. In another embodiment the pressure sensor is an optical sensor which is connected to an FFR processor via optical cables. In still further embodiments the present invention may be embodied by using any kind of pressure sensor arranged at the distal end of a pressure measurement device provided that the distal end of the device has a sufficiently small dimension making it possible to insert it into the vessel of interest.

When the severity of a lesion has been identified the treatment of the area may be by any of the usual therapeutic procedures, including localised delivery of a therapeutic agent, delivery of a stent, brachy therapy, ablation of selected tissue etc. Furthermore, the pressure sensor guidewire may additionally comprise angioplasty balloons or sleeves.

### SHORT DESCRIPTION OF THE APPENDED DRAWINGS

FIG. 1 is a block diagram schematically illustrating the present invention.

FIG. 2 is a block diagram schematically illustrating a first embodiment of the present invention.

FIG. 3 is a block diagram schematically illustrating a second embodiment of the present invention.

FIG. 4 is a block diagram schematically illustrating an embodiment of the present invention.

FIG. 5 is a diagram and a drawing illustrating the present invention.

FIG. 6 is a flow diagram illustrating the present invention.

FIG. 7 is a flow diagram illustrating an embodiment of the present invention.

### DETAILED DESCRIPTION OF PREFERRED EMBODIMENTS OF THE INVENTION

The present invention will now be described in detail with references to the appended drawings.

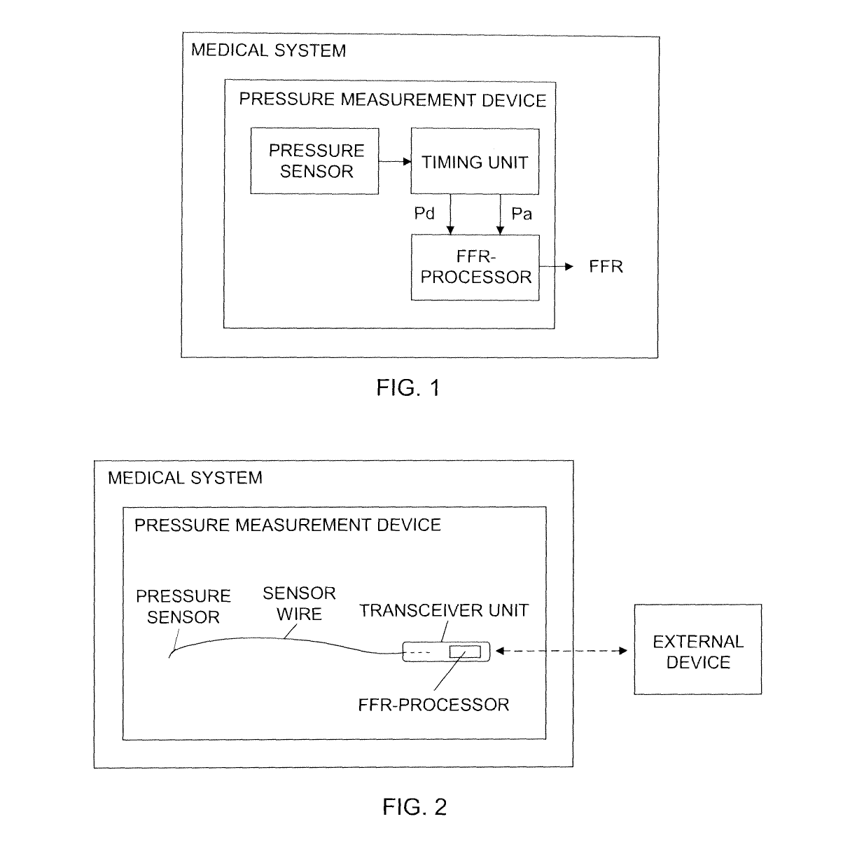

FIG. 1 is a block diagram illustrating a medical system, according to the present invention, for determining the individual Fractional Flow Reserve (FFR) value for one or many lesions of interest of a blood vessel. The system comprises an intravascular pressure measurement device for acquiring pressure measurements in the blood vessel, e.g. a coronary vessel, during continuous blood flow there through. The pressure measurement device comprises a pressure sensor at its distal portion. The system further comprises an FFR processor adapted to determine the FFR value related to said lesion solely based upon the pressure measurements performed by the pressure sensor.

The pressure measurement device further comprises a timing unit adapted to control the timing of the pressure measurements and the timing unit is adapted to control the pressure measurements such that a first pressure value (Pd) is measured distally said lesion, and a second pressure value (Pa) is measured proximally the lesion close to the aorta. Herein, the control of the pressure measurements should be interpreted as to ensure that corresponding first and second pressure values, Pd and Pa, respectively are related to each other, i.e. obtained from the same measurement session (pullback procedure). The pullback procedure, which is to be discussed below, ends when it is determined that the pressure sensor is in the aorta which may be visible at X-ray when the X-ray opaque distal end with the sensor element is just outside the opening of a guiding catheter placed in aorta.

The first pressure value (Pd) is measured at a first point of time t1 and the second pressure value (Pa) is measured at a second point of time t2, and that the time difference t2−t1 is greater than a first predetermined time value but less than a second predetermined time value. The time difference is typically in the interval of 5-10 seconds.

The FFR-processor preferably includes a memory where the measured pressure values are stored such that related values, i.e. values from the same measurement session, may be retrieved. The FFR-processor may further include a calculating unit for calculating the FFR value, i.e. forming the quotient between related values of Pd and Pa.

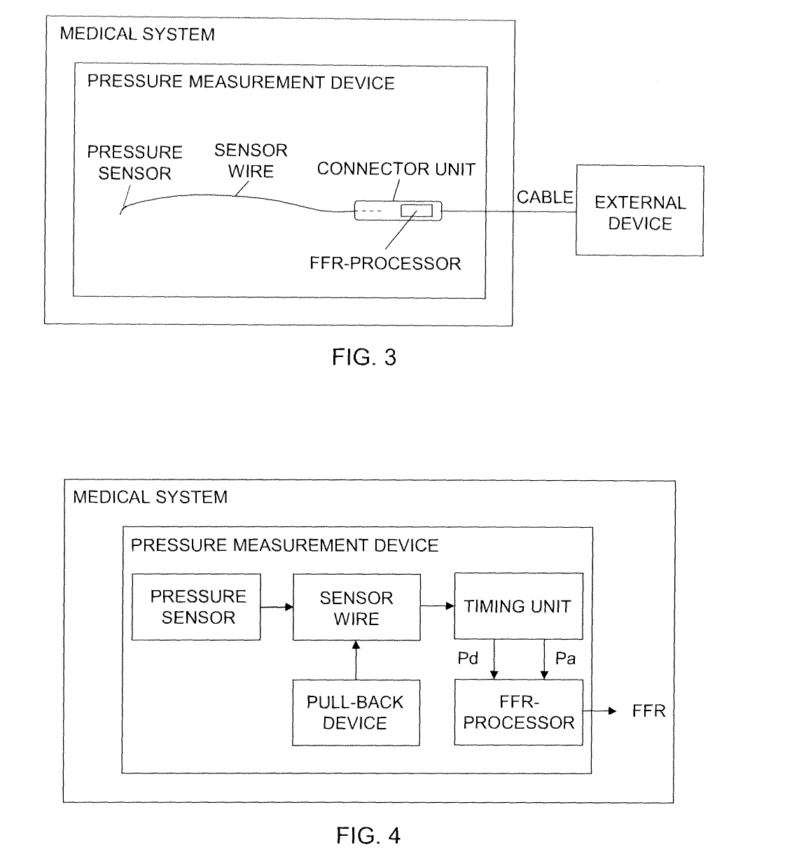

As described in the background section the intravascular pressure measurement device preferably may comprise an elongated sensor wire having an outer diameter of 0.3-0.5 mm and provided with the pressure sensor at its distal end portion, and is further provided with a proximal connector at its proximal end. This is schematically illustrated in FIGS. 2 and 3. As an alternative, as also discussed in the background section, the pressure measurement device may comprise a catheter, or a rapid-exchange catheter, provided with the pressure sensor at its distal end portion.

According to an embodiment of the invention, which is illustrated by FIG. 4, the pressure measurement device comprises a pull-back device adapted to pull-back the sensor wire from a first position (P1) where the pressure sensor senses a first pressure value (Pd) to a second position (P2) where the pressure sensor senses a second pressure value (Pa). The pull-back speed may be adjusted such that the pull-back procedure lasts less than a second predetermined time value, which preferably is in the interval of 5-10 seconds.

Whilst in normal circumstances the sensor guide wire provided with a pressure sensor is inserted manually, it is intended that when performing vascular measurements the pressure sensor guide wire is pulled back relative to a predetermined start position, preferably by using an electro-mechanical pull-back device e.g. coupled directly, or indirectly, to the sensor wire. EP-1291034 discloses a typical pull-back mechanism that may be used in connection with the sensor guide wire when implementing the present invention. The sensor guide wire is inserted such that a start position is reached when the pressure sensor is positioned distally the lesion to be measured.

The pull-back device may be controlled by a processing means (not shown) via a pull-back device interface (not shown). The system software accesses user-defined configuration files to get the necessary information about controlling the pull-back interface. Data sampling rate, recording duration and pre-selected retraction rate are taken into consideration for adjusting the pull-back speed. The speed may naturally be varied in dependence of the specific situation but as a general rule the speed is adjusted such that the pull-back procedure lasts for approximately 5-10 seconds.

The medical system according to the present invention may also be applied by manually pulling back the sensor wire (or catheter) from a first position (P1) where the pressure sensor senses a first pressure value (Pd) to a second position (P2) where the pressure sensor senses a second pressure value (Pa). The pull-back speed may such that the pull-back procedure lasts less than a second predetermined time value, which preferably is in the interval of 5-10 seconds.

FIG. 2 is a block diagram schematically illustrating a first embodiment of the present invention. According to the first embodiment the measurement device further comprises a transceiver unit into which the proximal connector is adapted to be inserted and attached, and that the transceiver unit comprises the FFR-processor. Preferably the transceiver unit comprises a communication module (not shown in the figure) adapted to wirelessly transfer the FFR-value to an external device. The transceiver unit may include a presentation unit for displaying said FFR-value.

As an alternative, the FFR-processor is arranged remote from the pressure measurement device, e.g. in an external device, and the detected pressure values, Pa and Pd, are transmitted to the FFR-processor for further processing.

FIG. 3 is a block diagram schematically illustrating a second embodiment of the present invention. According to the second embodiment the measurement device further comprises a connector unit into which the proximal connector of the sensor wire is adapted to be inserted and attached. The connector unit comprises the FFR-processor. The connector unit comprises a communication cable adapted to transfer the FFR-value to an external device, and that the connector unit may comprise a presentation unit for displaying the FFR-value.

As an alternative, the FFR-processor is arranged remote from the pressure measurement device, e.g. in an external device, and the detected pressure values, Pa and Pd, are transmitted, via the cable, to the FFR-processor for further processing.

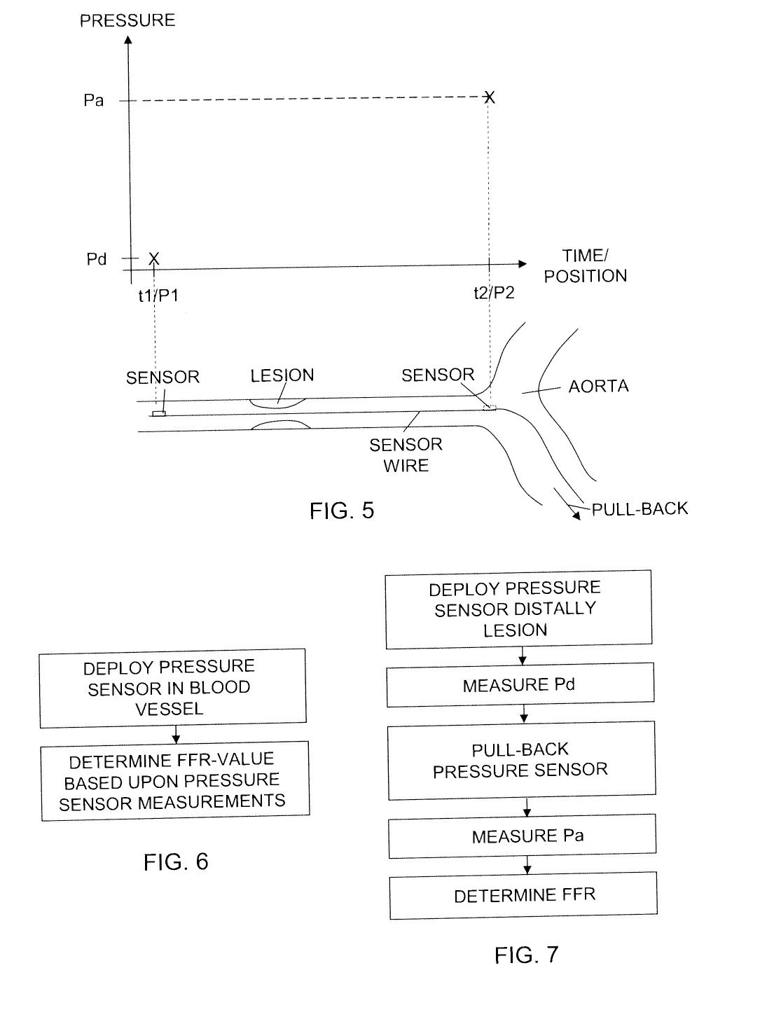

FIG. 5 is a diagram and a drawing illustrating the present invention.

The graph, above in FIG. 5, shows the pressure in relation to time, or in relation to the position of the pressure sensor.

Below in FIG. 5 is shown a simplified drawing of a coronary vessel and aorta, and a sensor wire provided with a pressure sensor inserted into the vessel such that the pressure sensor is positioned distally a lesion. In the position P1, the pressure sensed by the pressure sensor is Pd, which is denoted by an “X” in the graph.

The pressure sensor is then pulled back to position P2. The pullback is started at the time t1 and ends at time t2.

In position P2, i.e. when the pressure sensor is in the aorta, or close to where the coronary vessel opens into the aorta, the pressure sensor senses the pressure, which is Pa, and which also is denoted by an “X” in the graph.

In some occasions it might be of interest to monitor how the pressure varies in the coronary vessel during the entire, or during parts of, the pullback procedure. This pressure profile could then be presented in the graph as a curve between Pd and Pa.

In the graph the aortic pressure Pa is denoted by a straight dashed line which essentially represents the mean aortic pressure. The aortic pressure naturally changes in dependence of the pumping action of the heart.

The present invention also relates to a method for determining the individual Fractional Flow Reserve (FFR) value for one or many lesions of interest of a blood vessel. The method is schematically illustrated by the flow diagram of FIG. 6.

The method comprising:

a) deploying an intravascular pressure measurement device, provided with a pressure sensor, for acquiring pressure measurements in the blood vessel during continuous blood flow there through,

b) determining the FFR value related to said lesion solely based upon the pressure measurements performed by said pressure sensor.

With references to FIG. 7 the method in particular comprises controlling the timing of the pressure measurements such that a first pressure value (Pd) is measured distally the lesion, and a second pressure value (Pa) is measured proximally the lesion close to Aorta. The first pressure value (Pd) is measured at a first point of time t1 and the second pressure value (Pa) is measured at a second point of time t2, and that the time difference t2−t1 is greater than a first predetermined time value but less than a second predetermined time value. Preferably, these time values are 5 seconds and 10 seconds, respectively. The method comprises pulling back the pressure sensor from a first position where the pressure sensor senses the first pressure value (Pd) to a second position where the pressure sensor senses the second pressure value (Pa) and that the pull-back speed is adjusted such that the pull-back procedure lasts less than the second predetermined time value, which preferably is in the interval of 5-10 s.

It should be noted that the described method is equally applicable as a manual method or as a method where a pull-back device is used. If a manual method is used the physician manually pulls back the sensor wire. In that case the physician firstly inserts the sensor wire (or catheter) to a position where the pressure sensor is at a location distally the suspected lesion. Thereafter Pd is registered, e.g. automatically or by manually pressing a button or similar, and the sensor wire is pulled-back at a pull-back speed such that the second pressure value (Pa) may be registered, automatically or manually, 5-10 seconds after the registration of Pd. Then, the FFR-value is calculated using the registered Pd and Pa values as described above in connection with the description the medical system.

The present invention is not limited to the above-described preferred embodiments. Various alternatives, modifications and equivalents may be used. Therefore, the above embodiments should not be taken as limiting the scope of the invention, which is defined by the appending claims.

1. A medical system for determining the individual Fractional Flow Reserve (FFR) value for one or many lesions of interest of a blood vessel, the system comprising:
an intravascular pressure measurement device for acquiring pressure measurements in the blood vessel during continuous blood flow there through, said pressure measurement device comprises a pressure sensor at its distal portion,
characterized in that said system further comprises
an FFR processor adapted to determine the FFR value related to said lesion solely based upon the pressure measurements performed by said pressure sensor.

2. The system according to claim 1, wherein said pressure measurement device further comprises a timing unit adapted to control the timing of the pressure measurements.

3. The system according to claim 2, wherein said timing unit is adapted to control the pressure measurements such that a first pressure value (Pd) is measured distally said lesion, and a second pressure value (Pa) is measured proximally said lesion close to aorta.

4. The system according to claim 3, wherein said first pressure value (Pd) is measured at a first point of time t1 and the second pressure value (Pa) is measured at a second point of time t2, and that the time difference t2−t1 is greater than a first predetermined time value but less than a second predetermined time value.

5. The system according to claim 1, wherein said intravascular pressure measurement device comprises an elongated sensor wire having an outer diameter of 0.3-0.5 mm and provided with said pressure sensor at its distal end portion, and is further provided with a proximal connector at its proximal end.

6. The system according to claim 1, wherein said intravascular pressure measurement device comprises an elongated catheter provided with said pressure sensor at its distal end portion, and is further provided with a proximal connector at its proximal end.

7. The system according to claim 5, wherein said pressure measurement device is adapted to be manually pulled-back from a first position (P1) where said pressure sensor senses a first pressure value (Pd) to a second position (P2) where the pressure sensor senses a second pressure value (Pa), and that said sensed pressure values Pd and Pd are adapted to be registered by said FFR-processor.

8. The system according to claim 5, wherein said pressure measurement device comprises a pull-back device adapted to pull-back said sensor wire from a first position (P1) where said pressure sensor senses a first pressure value (Pd) to a second position (P2) where the pressure sensor senses a second pressure value (Pa).

9. The system according to claim 8, wherein a pull-back speed is adjusted at said pull-back device such that the pull-back procedure lasts less than a second predetermined time value.

10. The system according to claim 9, wherein said second predetermined time value is in the interval 5-10 s.

11. The system according to claim 5, wherein the measurement device further comprises a transceiver unit into which said proximal connector is adapted to be inserted and attached, said transceiver unit comprises said FFR-processor.

12. The system according to claim 11, wherein said transceiver unit comprises a communication module adapted to wirelessly transfer said FFR-value to an external device.

13. The system according to claim 11, wherein said transceiver unit comprises a presentation unit for displaying said FFR-value.

14. The system according to claim 5, wherein the measurement device further comprises a connector unit into which said proximal connector is adapted to be inserted and attached, said connector unit comprises said FFR-processor.

15. The system according to claim 14, wherein said connector unit comprises a communication cable adapted to transfer said FFR-value to an external device.

16. The system according to claim 14, wherein said connector unit comprises a presentation unit for displaying said FFR-value.

17. A method for determining the individual Fractional Flow Reserve (FFR) value for one or many lesions of interest of a blood vessel, the method comprising:
a) deploying an intravascular pressure measurement device, provided with a pressure sensor, for acquiring pressure measurements in the blood vessel during continuous blood flow there through,
b) determining the FFR value related to said lesion solely based upon the pressure measurements performed by said pressure sensor.

18. The method according to claim 17, wherein the method comprises controlling the timing of the pressure measurements such that a first pressure value (Pd) is measured distally said lesion, and a second pressure value (Pa) is measured proximally said lesion close to Aorta.

19. The method according to claim 18, wherein said first pressure value (Pd) is measured at a first point of time t1 and the second pressure value (Pa) is measured at a second point of time t2, and that the time difference t2−t1 is greater than a first predetermined time value but less than a second predetermined time value.

20. The method according to claim 17, wherein the method comprises pulling back said pressure sensor from a first position where said pressure sensor senses a first pressure value (Pd) to a second position where the pressure sensor senses a second pressure value (Pa).

21. The method according to claim 20, wherein a pull-back speed is adjusted such that the pull-back procedure lasts less than a second predetermined time value.

22. The method according to claim 21, wherein said second predetermined time value is in the interval 5-10 s.
